SEQUENCE LISTING (72 sequences)

SEQ ID NO: 1 - DNA, 23 bp, Homo sapiens
uacccuguagauccgaauuugug

SEQ ID NO: 2 - DNA, 23 bp, Homo sapiens
uacccuguagaaccgaauuugug

SEQ ID NO: 3 - DNA, 22 bp, Homo sapiens
uagcagcacauaaugguuugug

SEQ ID NO: 4 - DNA, 22 bp, Homo sapiens
uagcagcacaucaugguuuaca

SEQ ID NO: 5 - DNA, 22 bp, Homo sapiens
uagcagcacguaaauauuggcg

SEQ ID NO: 6 - DNA, 23 bp, Homo sapiens
caaagugcuuacagugcagguag

SEQ ID NO: 7 - DNA, 23 bp, Homo sapiens
ugugcaaaucuaugcaaaacuga

SEQ ID NO: 8 - DNA, 23 bp, Homo sapiens
ugugcaaauccaugcaaaacuga

SEQ ID NO: 9 - DNA, 23 bp, Homo sapiens
uaaagugcuuauagugcagguag

SEQ ID NO: 10 - DNA, 23 bp, Homo sapiens
caaagugcucauagugcagguag

SEQ ID NO: 11 - DNA, 22 bp, Homo sapiens
uagcuuaucagacugauguuga

SEQ ID NO: 12 - DNA, 22 bp, Homo sapiens
aagcugccaguugaagaacugu

SEQ ID NO: 13 - DNA, 21 bp, Homo sapiens
aucacauugccagggauuucc

SEQ ID NO: 14 - DNA, 21 bp, Homo sapiens
aucacauugccagggauuacc

SEQ ID NO: 15 - DNA, 22 bp, Homo sapiens
uggcucaguucagcaggaacag

SEQ ID NO: 16 - DNA, 22 bp, Homo sapiens
uucaaguaauccaggauaggcu

SEQ ID NO: 17 - DNA, 21 bp, Homo sapiens
uucaaguaauucaggauaggu

SEQ ID NO: 18 - DNA, 21 bp, Homo sapiens
uucacaguggcuaaguuccgc

SEQ ID NO: 19 - DNA, 21 bp, Homo sapiens
uucacaguggcuaaguucugc

SEQ ID NO: 20 - DNA, 22 bp, Homo sapiens
aaggagcucacagucuauugag

SEQ ID NO: 21 - DNA, 22 bp, Homo sapiens
uagcaccaucugaaaucgguua

SEQ ID NO: 22 - DNA, 22 bp, Homo sapiens
uagcaccauuugaaaucgguua

SEQ ID NO: 23 - DNA, 22 bp, Homo sapiens
uguaaacauccucgacuggaag

SEQ ID NO: 24 - DNA, 22 bp, Homo sapiens
uguaaacauccuacacucagcu

SEQ ID NO: 25 - DNA, 23 bp, Homo sapiens
uguaaacauccuacacucucagc

SEQ ID NO: 26 - DNA, 22 bp, Homo sapiens
uguaaacauccccgacuggaag

SEQ ID NO: 27 - DNA, 22 bp, Homo sapiens
uguaaacauccuugacuggaag

SEQ ID NO: 28 - DNA, 22 bp, Homo sapiens
uauugcacuugucccggccugu

SEQ ID NO: 29 - DNA, 23 bp, Homo sapiens
caaagugcuguucgugcagguag

SEQ ID NO: 30 - DNA, 22 bp, Homo sapiens
ugagguaguaaguuguauuguu

SEQ ID NO: 31 - DNA, 22 bp, Homo sapiens
aacccguagauccgaucuugug

SEQ ID NO: 32 - DNA, 22 bp, Homo sapiens
cacccguagaaccgaccuugcg

SEQ ID NO: 33 - DNA, 22 bp, Homo sapiens
aacccguagauccgaacuugug

SEQ ID NO: 34 - DNA, 21 bp, Homo sapiens
uacaguacugugauaacugaa

SEQ ID NO: 35 - DNA, 23 bp, Homo sapiens
agcagcauuguacagggcuauga

SEQ ID NO: 36 - DNA, 23 bp, Homo sapiens
aaaagugcuuacagugcagguag

SEQ ID NO: 37 - DNA, 21 bp, Homo sapiens
uaaagugcugacagugcagau

SEQ ID NO: 38 - DNA, 23 bp, Homo sapiens
agcagcauuguacagggcuauca

SEQ ID NO: 39 - DNA, 24 bp, Homo sapiens
ucccugagacccuuuaaccuguga

SEQ ID NO: 40 - DNA, 22 bp, Homo sapiens
ucguaccgugaguaauaaugcg

SEQ ID NO: 41 - DNA, 21 bp, Homo sapiens
cauuauuacuuuugguacgcg

SEQ ID NO: 42 - DNA, 22 bp, Homo sapiens
cagugcaauguuaaaagggcau

SEQ ID NO: 43 - DNA, 22 bp, Homo sapiens
uuugguccccuucaaccagcug

SEQ ID NO: 44 - DNA, 22 bp, Homo sapiens
uuugguccccuucaaccagcua

SEQ ID NO: 45 - DNA, 21 bp, Homo sapiens
ugagaugaagcacuguagcuc

SEQ ID NO: 46 - DNA, 22 bp, Homo sapiens
ugagaacugaauuccauggguu

SEQ ID NO: 47 - DNA, 22 bp, Homo sapiens
ugagaacugaauuccauaggcu

SEQ ID NO: 48 - DNA, 22 bp, Homo sapiens
ucucccaacccuuguaccagug

SEQ ID NO: 49 - DNA, 21 bp, Homo sapiens
ucagugcaugacagaacuugg

SEQ ID NO: 50 - DNA, 23 bp, Homo sapiens
aacauucaacgcugucggugagu

SEQ ID NO: 51 - DNA, 22 bp, Homo sapiens
uggagagaaaggcaguuccuga

SEQ ID NO: 52 - DNA, 23 bp, Homo sapiens
caacggaaucccaaaagcagcug

SEQ ID NO: 53 - DNA, 22 bp, Homo sapiens
gcugcgcuuggauuucgucccc

SEQ ID NO: 54 - DNA, 21 bp, Homo sapiens
uagcagcacagaaauauuggc

SEQ ID NO: 55 - DNA, 22 bp, Homo sapiens
auaagacgagcaaaaagcuugu

SEQ ID NO: 56 - DNA, 21 bp, Homo sapiens
agcuacaucuggcuacugggu

SEQ ID NO: 57 - DNA, 22 bp, Homo sapiens
aaaagcuggguugagagggcga

SEQ ID NO: 58 - DNA, 23 bp, Homo sapiens
ucaagagcaauaacgaaaaaugu

SEQ ID NO: 59 - DNA, 22 bp, Homo sapiens
uaaugccccuaaaaauccuuau

SEQ ID NO: 60 - DNA, 22 bp, Homo sapiens
uuauaauacaaccugauaagug

SEQ ID NO: 61 - DNA, 22 bp, Homo sapiens
cagcagcaauucauguuuugaa

SEQ ID NO: 62 - DNA, 22 bp, Homo sapiens
aaaccguuaccauuacugaguu

SEQ ID NO: 63 - DNA, 22 bp, Homo sapiens
aaacaaacauggugcacuucuu

SEQ ID NO: 64 - DNA, 22 bp, Homo sapiens
uggaauguaaagaaguauguau

SEQ ID NO: 65 - DNA, 23 bp, Homo sapiens
uggaagacuagugauuuuguugu

SEQ ID NO: 66 - DNA, 23 bp, Homo sapiens
uagcaccauuugaaaucaguguu

SEQ ID NO: 67 - DNA, 22 bp, Homo sapiens
ucccugagacccuaacuuguga

SEQ ID NO: 68 - DNA, 23 bp, Homo sapiens
guccaguuuucccaggaaucccu

SEQ ID NO: 69 - DNA, 23 bp, Homo sapiens
aacauucauugcugucggugggu

SEQ ID NO: 70 - DNA, 22 bp, Homo sapiens
acagcaggcacagacaggcagu

SEQ ID NO: 71 - DNA, 21 bp, Homo sapiens
aggggugcuaucugugauuga

SEQ ID NO: 72 - DNA, 21 bp, Homo sapiens
acuggacuuggagucagaagg

(19) United States
(12) Patent Application Publication
(10) Pub. No.: US 2011/0086348 A1
(43) Pub. Date: Apr. 14, 2011

(54) METHOD FOR ASSESSING HEART DISEASE

(75) Inventors: Sathyamangla V. Naga Prasad, Beachwood, OH (US); Sadashiva Karnik, Shaker Heights, OH (US); Carlo Croce, Columbus, OH (US); Dianne M. Perez, Broadview Heights, OH (US); Edward Plow, Solon, OH (US); Christine Moravec, Shaker Heights, OH (US); Subha Sen, Solon, OH (US); Qingyu Wu, Orange Village, OH (US); Randall C. Starling, Chagrin Falls, OH (US)

(73) Assignee: The Cleveland Clinic Foundation

(21) Appl. No.: 12/709,010

(22) Filed: Feb. 19, 2010

Related U.S. Application Data
(60) Provisional application No. 61/153,856, filed on Feb. 19, 2009.

(51) Int. Cl. C12Q 1/68

(52) U.S. Cl. 435/6

(57) ABSTRACT

A method for assessing heart disease in a subject includes generating an expression profile of at least two or more miRNAs in a sample from the subject. The miRNAs can be selected from the group consisting of hsa-miRNA-1, hsa-miRNA-7, hsa-miRNA-29b, has-miRNA-125b, hsa-miRNA145, hsa-miRNA-181b, hsa-miRNA-214, hsa-miRNA-342, hsa-miRNA-378 and combinations thereof.

### RELATED APPLICATION

This application claims priority from U.S. Provisional Patent Application Ser. No. 61//153,856, filed Feb. 19, 2009, the entirety of which is incorporated herein by reference.

### TECHNICAL FIELD

The present invention relates generally to a method of assessing heart disease, and specifically relates to methods of diagnosis and prognosis of heart failure by measuring the expression level of a subject's miRNA.

### BACKGROUND OF THE INVENTION

The increase in heart failure (HF) has reached epidemic proportions in the US, with an estimated 550,000 new cases each year. As patients survive various forms of heart disease due to improved therapies, HF has become more prevalent. Currently, the only “cure” for HF is transplantation, but the number of patients needing transplants far outstrips the availability of donor hearts. Many drugs and therapeutic regimens are available to delay the progression of HF. The success of such therapies depends on early diagnosis and ways to monitor the therapeutic response, which varies greatly among the individual patients. Therefore, there is a need for identifying cardiac dysfunction early in onset and monitoring the response to therapy in order to better treat patients with heart failure.

The recent classification statement from the American Heart Association (AHA)/America College of Cardiology (ACC) substantiates the need for early diagnosis of HF. This classification identifies a much broader range of stages (Stages A to D) in the evolution of HF. It includes early stages A and B, placed ahead of the stages in the New York Heart Association (NYHA) function based classification (Classes I though IV), which had long been the standard in the field. It is contemplated that primary prevention of HF by intervention at stages A and B could lead to favorable HF prognosis. Therefore, successful prevention of HF hinges on the ability to identify molecular changes early-on at the onset (stages A & B) to initiate appropriate medical therapy. Furthermore, with an increasing emphasis on early clinical intervention, the need for effective tools to monitor responses is clear. Currently, the most commonly used assays to diagnose HF and monitor HF therapy are ANP/BNP assays which fail to detect early HF and frequently give false positive or false negative results. Thus, sensitive diagnostic tools for early detection are integral to successful treatment of HF.

### SUMMARY OF THE INVENTION

The present invention relates to methods and compositions of assessing heart disease in a subject. The methods can be used to diagnose heart disease, assess the progress of heart disease, and monitor the response of heart disease to therapy in the subject, including those subjects with early-stage heart failure.

The method can include obtaining a biological sample from the subject that includes miRNA. The expression level of at least two miRNAs selected from the group consisting of hsa-miRNA-1, hsa-miRNA-7, hsa-miRNA-29b, hsa-miRNA-125b, has-miRNA-145, hsa-miRNA-181b, hsa-miRNA-214, hsa-miRNA-342, and hsa-miRNA-378 can be measured in the sample. The measured expression level of the at least two miRNAs can then be compared to a control expression level in normal heart tissue. A difference in the expression level of the sample from the subject and the control expression level is indicative of heart disease.

In an aspect of the invention, the biological sample can include myocardial tissue or myocardial cells that are obtained from subject by biopsy. The biological sample can also include blood from the subject. In a particular aspect of the invention, the biological sample can be peripheral blood mononuclear cells that are isolated from the blood.

In another aspect of the invention, a down-regulated expression level of at least one miRNA comprising hsa-mir-001, hsa-mir-007, hsa-mir-29b, or hsa-mir-378 compared to the control expression profile is indicative of heart disease; and, an up-regulated expression level of at least one miRNA comprising hsa-mir-125b, hsa-mir-181b, hsa-mir-214, or has-mir-342 compared to the control expression profile is indicative of heart disease.

In a further aspect, the expression level of at least two miRNAs can be determined by an amplification assay. Alternatively, the expression profile of at least two miRNAs being determined by a hybridization assay.

The present invention also relates to a method for assessing heart disease in a subject that includes obtaining a sample of peripheral blood mononuclear cells from the subject. The expression levels of at least one miRNA associated with heart disease is measured from the peripheral blood mononuclear cell sample. The expression of the at least one miRNA from the peripheral blood mononuclear cell sample can correspond to miRNA expression level in the subject's myocardial tissue. The expression levels of the measured miRNA are then compared to a control expression level. A difference in the measured expression level and the control expression level is indicative of heart disease.

In an aspect of the invention, the expression levels of miRNAs measured are selected from the group consisting of hsa-miRNA-1, hsa-miRNA-7, hsa-miRNA-29b, has-miRNA-125b, hsa-miRNA-145, hsa-miRNA-181b, hsa-miRNA-214, hsa-miRNA-342, hsa-miRNA-378 and combinations thereof a down-regulated expression level of at least one miRNA comprising hsa-mir-001, hsa-mir-007, hsa-mir-29b, or hsa-mir-378 compared to the control expression profile is indicative of heart disease; and, an up-regulated expression level of at least one miRNA comprising hsa-mir-125b, hsa-mir-181b, hsa-mir-214, or hsa-mir-342 compared to the control expression profile is indicative of heart disease.

### BRIEF DESCRIPTION OF THE DRAWINGS

The foregoing and other features and advantages of the present invention will become apparent to those skilled in the art to which the present invention relates upon reading the following description with reference to the accompanying drawings, in which:

FIG. 1 illustrates a flow diagram of a method of determining miRNA associated with dilated cardiomyopathy.

FIG. 2 illustrates the expression values for differentially expressed miRNA probes in accordance with the present invention.

FIG. 3 is illustrates graphs comparing the expression levels of selected miRNA levels in non-failing and dilated cardiomyopathy human patient samples.

FIG. 4 illustrates a schematic network of predicted targets for selected miRNA in accordance with the present invention.

FIG. 5 illustrates a graph showing the expression levels of selected miRNA in peripheral blood mononuclear cells (PBMCs) of subjects with heart failure compared to control.

### DETAILED DESCRIPTION

Methods involving conventional molecular biology techniques are described herein. Such techniques are generally known in the art and are described in detail in methodology treatises, such as Molecular Cloning: A Laboratory Manual, 2nd ed., vol. 1-3, ed. Sambrook et al., Cold Spring Harbor Laboratory Press, Cold Spring Harbor, N.Y., 1989, and Current Protocols in Molecular Biology, ed. Ausubel et al., Greene Publishing and Wiley-Interscience, New York, 1992 (with periodic updates). Methods for chemical synthesis of nucleic acids are discussed, for example, in Beaucage and Carruthers, Tetra. Letts. 22:1859-1862, 1981, and Matteucci et al., J. Am. Chem. Soc. 103:3185, 1981. Chemical synthesis of nucleic acids can be performed, for example, on commercial automated oligonucleotide synthesizers. Conventional methods of gene transfer and gene therapy can also be adapted for use in the present invention. See, e.g. Gene Therapy: Principles and Applications, ed. T. Blackenstein, Springer Verlag, 1999, Gene Therapy Protocols (Methods in Molecular Medicine), ed. P. D. Robbins, Humana Press, 1997, and Retro-vectors for Human Gene Therapy, ed. C. P. Hodgson, Springer Verlag, 1996.

Unless otherwise defined, all technical terms used herein have the same meaning as commonly understood by one of ordinary skill in the art to which the present invention pertains. Commonly understood definitions of molecular biology terms can be found in, for example, Rieger et al., Glossary of Genetics: Classical and Molecular, 5th edition, Springer-Verlag: New York, 1991, and Lewin, Genes V, Oxford University Press: New York, 1994.

The present invention relates generally to a method and compositions of assessing heart disease in a human subject at risk and/or having heart disease. The method includes measuring the levels (increased or decreased) of selected miRNAs in patient-derived biological samples in order to create a miRNA expression profile. The measured miRNA levels used to generate an expression profile can be compared with a miRNA expression profile of a control sample (e.g., the miRNA expression profile of a sample obtained from a healthy subject or a subject free of heart disease) to determine an alteration and/or difference in miRNA expression levels between subject and the control. A difference in miRNA expression levels can be used to screen subjects for heart disease, monitor the progress of heart disease, assess the prognosis of heart disease, stage heart disease, and/or assess response of heart disease to therapy and/or treatments. Prognosis of heart disease can include, but is not limited to an estimation of the time or expected time of survival, assessment of response to a therapy, and the like.

The term “miRNA” is used according to its ordinary and plain meaning and refers to a microRNA molecule found in eukaryotes that is involved in RNA-based gene regulation. The term can be used to refer to the single-stranded RNA molecule processed from a precursor or in certain instances the precursor itself. Individual miRNAs have been identified and sequenced in different organisms, and they have been given names.

In some embodiments, “miRNA” molecules implemented in the invention will also encompass a region or an additional strand that is partially (between 10 and 50% complementary across length of strand), substantially (greater than 50% but less than 100% complementary across length of strand) or fully complementary to another region of the same single stranded molecule or to another nucleic acid. Thus, nucleic acids may encompass a molecule that comprises one or more complementary or self-complementary strand(s) or “complements” of a particular sequence comprising a molecule. For example, precursor miRNA may have a self-complementary region, which is up to 100% complementary miRNA probes of the invention can be or be at least 60, 65, 70, 75, 80, 85, 90, 95 or 100% complementary to their target.

The term “miRNA expression profile” refers to a set of data regarding the expression pattern of at least one miRNA (e.g. a plurality of miRNA from TABLE 1 and TABLE 2) in the sample. It is contemplated that the miRNA expression profile can be generated or obtained using a set of two or more miRNAs, using for example nucleic acid amplification or hybridization techniques well know to one of ordinary skill in the art. The difference in the expression profile in the sample from the patient and a reference expression profile, such as an expression profile from a normal or non-pathologic sample, can be indicative of heart disease.

In certain embodiments, miRNA expression profiles may be generated to evaluate and correlate those profiles with pharmacokinetics. For example, miRNA expression profiles may be created and evaluated prior to the subjects' being treated or during treatment to determine if there are miRNAs whose expression correlates with the outcome of the patient's treatment. The miRNA profiles described herein can be used for evaluation of tissue and/or blood samples to determine what drug regimen the subjects should be provided. In addition, the miRNA expression profiles can be used to identify or select subjects suitable for a particular clinical trial. An miRNA expression profile determined to correlate with drug efficacy or drug toxicity may be relevant to whether that patient is an appropriate patient for receiving the drug or for a particular dosage of the drug.

One particular type of heart disease contemplated by the present invention is early-stage heart failure. One aspect of the present invention provides a method of assessing early-stage heart failure. The term “early-stage heart failure” can refer to the classification statement from the American Heart Association (AHA)/America College of Cardiology (ACC), which identifies a range of stages (Stages A to D) in the evolution of HF. It includes early stages A and B, placed ahead of the stages in the New York Heart Association (NYHA) function based classification (Classes I though IV), which had long been the standard in the field.

The miRNA that is measured in the methods of the present invention can be obtained from a biological sample of a subject that is assessed for heart disease. The biological sample can include tissue or cells in which the miRNA expression profile is indicative of whether the subject is at risk and/or has heart disease. In one aspect of the invention, the biological sample can include myocardial tissue or myocardial cells, such myocardial tissue and/or cells obtained by biopsy.

In another aspect of the invention, the biological sample can include peripheral blood mononuclear cells (PBMCs) isolated from blood of the subject. It was unexpectedly found that the expression levels of miRNAs obtained from a PBMC sample of a subject directly corresponds to a miRNA expression profile of the subject's myocardial tissue. It is believed that the circulatory system (including cells) maintains an active dialog with the heart. This active dialog allows the PBMCs to reflect the cellular/molecular changes occurring in the heart, even when ventricular systolic function is still preserved. The cellular/molecular changes can be assessed and/or monitored by measuring the miRNA expression levels in the PBMCs. This is advantageous as the PBMCs, unlike myocardial tissue or cells, can be obtained by minimally invasive means from the subject. Thus, one aspect of the invention relates to a non- or minimally invasive method of assessing heart disease in a subject my measuring the expression levels of miRNAs associated with heart disease in PBMCs obtained from the subject and comparing the measured miRNA levels with control miRNA levels, such as comparative miRNA levels of PBMCs from a normal healthy subject.

Advantageously, miRNA levels can be measured in PBMCs obtained from the subject at various instances over a duration of time (e.g. days, weeks, months, years). These measured miRNA levels can be compared to previous measured miRNA levels of the subject and/or miRNA levels of healthy controls to monitor the progress of heart disease and/or measure the efficacy of a therapeutic treatment.

In an aspect of the invention, the PBMCs can be obtained from the subject by extracting the PBMCs from a sample of whole blood obtained from the subject. By way of example, the PBMCs can be extracted from blood using ficoll, a hydrophilic polysaccharide that separates layers of blood, with monocytes and lymphocytes forming a buffy coat under a layer of plasma. This buffy coat contains the PBMCs. Additionally, PBMC can be extracted from whole blood using a hypotonic lysis, which will preferentially lyse red blood cells. Other known methods of obtaining or isolating PBMCs include those described in U.S. Patent Application Publication No. 2007/0281352, which is herein incorporated by reference in its entirety.

The miRNA levels measured in the biological sample, which can be used to generate an expression profile, can include any miRNA whose altered expression is associated with cardiovascular disease. Examples of miRNA whose altered expression is associated with heart disease can include those identified below in TABLE 1.

TABLE 1

| PROBE SEQUENCE | SEQ ID NO: | miRNA | miR Base Information |
|---|---|---|---|
| UACCCUGUAGAUCCGAAUUUGUG | SEQ ID NO: 1 | hsa-mir-10a | MIMAT0000253 |
| UACCCUGUAGAACCGAAUUUGUG | SEQ ID NO: 2 | hsa-mir-10b | MIMAT0000254 |
| UAGCAGCACAUAAUGGUUUGUG | SEQ ID NO: 3 | hsa-mir-15a | MIMAT0000068 |
| UAGCAGCACAUCAUGGUUUACA | SEQ ID NO: 4 | hsa-mir-15b | MIMAT0000417 |
| UAGCAGCACGUAAAUAUUGGCG | SEQ ID NO: 5 | hsa-mir-16 | MIMAT0000069 |
| CAAAGUGCUUACAGUGCAGGUAG | SEQ ID NO: 6 | hsa-mir-17-5p | MIMAT0000070 |
| UGUGCAAAUCUAUGCAAAACUGA | SEQ ID NO: 7 | hsa-mir-19a | MIMAT0000073 |
| UGUGCAAAUCCAUGCAAAACUGA | SEQ ID NO: 8 | hsa-mir-19b | MIMAT0000074 |
| UAAAGUGCUUAUAGUGCAGGUAG | SEQ ID NO: 9 | hsa-mir-20a | MIMAT0000075 |
| CAAAGUGCUCAUAGUGCAGGUAG | SEQ ID NO: 10 | hsa-mir-20b | MIMAT0001413 |
| UAGCUUAUCAGACUGAUGUUGA | SEQ ID NO: 11 | hsa-mir-21 | MIMAT0000076 |
| AAGCUGCCAGUUGAAGAACUGU | SEQ ID NO: 12 | hsa-mir-22 | MIMAT0000077 |
| AUCACAUUGCCAGGGAUUUCC | SEQ ID NO: 13 | hsa-mir-23a | MIMAT0000078 |
| AUCACAUUGCCAGGGAUUACC | SEQ ID NO: 14 | hsa-mir-23b | MIMAT0000418 |
| UGGCUCAGUUCAGCAGGAACAG | SEQ ID NO: 15 | hsa-mir-24 | MIMAT0000080 |
| UUCAAGUAAUCCAGGAUAGGCU | SEQ ID NO: 16 | hsa-mir-26a | MIMAT0000082 |
| UUCAAGUAAUUCAGGAUAGGU | SEQ ID NO: 17 | hsa-mir-26b | MIMAT0000083 |
| UUCACAGUGGCUAAGUUCCGC | SEQ ID NO: 18 | hsa-mir-27a | MIMAT0000084 |
| UUCACAGUGGCUAAGUUCUGC | SEQ ID NO: 19 | hsa-mir-27b | MIMAT0000419 |
| AAGGAGCUCACAGUCUAUUGAG | SEQ ID NO: 20 | hsa-mir-28 | MIMAT0000085 |
| UAGCACCAUCUGAAAUCGGUUA | SEQ ID NO: 21 | hsa-mir-29a | MIMAT0000086 |
| UAGCACCAUUUGAAAUCGGUUA | SEQ ID NO: 22 | hsa-mir-29c | MIMAT0000681 |
| UGUAAACAUCCUCGACUGGAAG | SEQ ID NO: 23 | hsa-mir-30a | MIMAT0000087 |
| UGUAAACAUCCUACACUCAGCU | SEQ ID NO: 24 | hsa-mir-30b | MIMAT0000420 |
| UGUAAACAUCCUACACUCUCAGC | SEQ ID NO: 25 | hsa-mir-30c | MIMAT0000244 |
| UGUAAACAUCCCCGACUGGAAG | SEQ ID NO: 26 | hsa-mir-30d | MIMAT0000245 |
| UGUAAACAUCCUUGACUGGAAG | SEQ ID NO: 27 | hsa-mir-30e | MIMAT0000692 |
| UAUUGCACUUGUCCCGGCCUGU | SEQ ID NO: 28 | hsa-mir-92 | MIMAT0000092 |
| CAAAGUGCUGUUCGUGCAGGUAG | SEQ ID NO: 29 | hsa-mir-93 | MIMAT0000093 |
| UGAGGUAGUAAGUUGUAUUGUU | SEQ ID NO: 30 | hsa-mir-98 | MIMAT0000096 |
| AACCCGUAGAUCCGAUCUUGUG | SEQ ID NO: 31 | hsa-mir-99a | MIMAT0000097 |
| CACCCGUAGAACCGACCUUGCG | SEQ ID NO: 32 | hsa-mir-99b | MIMAT0000689 |
| AACCCGUAGAUCCGAACUUGUG | SEQ ID NO: 33 | hsa-mir-100 | MIMAT0000100 |
| UACAGUACUGUGAUAACUGAA | SEQ ID NO: 34 | hsa-mir-101 | MIMAT0000099 |
| AGCAGCAUUGUACAGGGCUAUGA | SEQ ID NO: 35 | hsa-mir-103 | MIMAT0000101 |
| AAAAGUGCUUACAGUGCAGGUAG | SEQ ID NO: 36 | hsa-mir-106a | MIMAT0000103 |
| UAAAGUGCUGACAGUGCAGAU | SEQ ID NO: 37 | hsa-mir-106b | MIMAT0000680 |
| AGCAGCAUUGUACAGGGCUAUCA | SEQ ID NO: 38 | hsa-mir-107 | MIMAT0000104 |
| UCCCUGAGACCCUUUAACCUGUGA | SEQ ID NO: 39 | hsa-mir-125a | MIMAT0000443 |
| UCGUACCGUGAGUAAUAAUGCG | SEQ ID NO: 40 | hsa-mir-126 | MIMAT0000445 |
| CAUUAUUACUUUUGGUACGCG | SEQ ID NO: 41 | hsa-mir-126* | MIMAT0000444 |
| CAGUGCAAUGUUAAAAGGGCAU | SEQ ID NO: 42 | hsa-mir-130a | MIMAT0000425 |
| UUUGGUCCCCUUCAACCAGCUG | SEQ ID NO: 43 | hsa-mir-130b | MIMAT0000427 |
| UUUGGUCCCCUUCAACCAGCUA | SEQ ID NO: 44 | hsa-mir-140* | MIMAT0000770 |
| UGAGAUGAAGCACUGUAGCUC | SEQ ID NO: 45 | hsa-mir-143 | MIMAT0000435 |
| UGAGAACUGAAUUCCAUGGGUU | SEQ ID NO: 46 | hsa-mir-146a | MIMAT0000449 |
| UGAGAACUGAAUUCCAUAGGCU | SEQ ID NO: 47 | hsa-mir-146b-5p | MIMAT0002809 |
| UCUCCCAACCCUUGUACCAGUG | SEQ ID NO: 48 | hsa-mir-150 | MIMAT0000451 |
| UCAGUGCAUGACAGAACUUGG | SEQ ID NO: 49 | hsa-mir-152 | MIMAT0000438 |
| AACAUUCAACGCUGUCGGUGAGU | SEQ ID NO: 50 | hsa-mir-181a | MIMAT0000256 |
| UGGAGAGAAAGGCAGUUCCUGA | SEQ ID NO: 51 | hsa-mir-185 | MIMAT0000455 |
| CAACGGAAUCCCAAAAGCAGCUG | SEQ ID NO: 52 | hsa-mir-191 | MIMAT0000440 |
| GCUGCGCUUGGAUUUCGUCCCC | SEQ ID NO: 53 | hsa-mir-191* | MIMAT0001618 |
| UAGCAGCACAGAAAUAUUGGC | SEQ ID NO: 54 | hsa-mir-195 | MIMAT0000461 |
| AUAAGACGAGCAAAAAGCUUGU | SEQ ID NO: 55 | hsa-mir-208 | MIMAT0000241 |
| AGCUACAUCUGGCUACUGGGU | SEQ ID NO: 56 | hsa-mir-222 | MIMAT0000279 |
| AAAAGCUGGGUUGAGAGGGCGA | SEQ ID NO: 57 | hsa-mir-320 | MIMAT0000510 |
| UCAAGAGCAAUAACGAAAAAUGU | SEQ ID NO: 58 | hsa-mir-335 | MIMAT0000765 |
| UAAUGCCCCUAAAAAUCCUUAU | SEQ ID NO: 59 | hsa-mir-365 | MIMAT0000710 |
| UUAUAAUACAACCUGAUAAGUG | SEQ ID NO: 60 | hsa-mir-374 | MIMAT0000727 |
| CAGCAGCAAUUCAUGUUUUGAA | SEQ ID NO: 61 | hsa-mir-424 | MIMAT0001341 |
| AAACCGUUACCAUUACUGAGUU | SEQ ID NO: 62 | hsa-mir-451 | MIMAT0001631 |
| AAACAAACAUGGUGCACUUCUU | SEQ ID NO: 63 | hsa-mir-495 | MIMAT0002817 |

It will be appreciated, that other miRNAs whose altered expression is associated with human heart disease can be used in the methods of the present invention, and that the miRNA used in the methods of the present invention need not be limited to the miRNAs used in TABLE 1. Examples of other miRNAs whose altered expression is associated with human heart disease are described in, for example, Ikeda et al. (2007) Physiol Genomics 31: 367-373, Latronico et al. (2008) Physiol Genomics. 15;34(3):239-42; and Sucharov et al. (2008) J Mol Cell Cardiol. August; 45(2):185-92. Additionally, any embodiment of the invention involving specific miRNAs by name is contemplated also to cover embodiments involving miRNAs whose sequences are at least 80, 81, 82, 83, 84, 85, 86, 87, 88, 89, 90, 91, 92, 93, 94, 95, 96, 97, 98, 99% identical to the mature sequence of the specified miRNA.

In accordance with another aspect of the invention, the levels of at least two miRNAs selected from the group consisting of hsa-miRNA-1, hsa-miRNA-7, has-miRNA-29b, hsa-miRNA-125b, hsa-miRNA145, hsa-miRNA-181b, hsa-miRNA-214, hsa-miRNA-342, and hsa-miRNA-378, which are shown in TABLE 2, can be measured in the biological sample to generate an expression profile to assess whether the subject has heart disease.

TABLE 2
Listing of miRNA probes for sample assessment.

| Probe Sequence | SEQ ID NO: | miRNA | miR Base Information |
|---|---|---|---|
| UGGAAUGUAAAGAAGUAUGUAU | SEQ ID NO: 64 | hsa-mir-1 | MIMAT0000416 |
| UGGAAGACUAGUGAUUUUGUUGU | SEQ ID NO: 65 | hsa-mir-7 | MIMAT0000252 |
| UAGCACCAUUUGAAAUCAGUGUU | SEQ ID NO: 66 | hs a-mir-29b | MIMAT0000100 |
| UCCCUGAGACCCUAACUUGUGA | SEQ ID NO: 67 | hsa-mir-125b | MIMAT0000423 |
| GUCCAGUUUUCCCAGGAAUCCCU | SEQ ID NO: 68 | hsa-mir-145 | MIMAT0000437 |
| AACAUUCAUUGCUGUCGGUGGGU | SEQ ID NO: 69 | hsa-mir-181b | MIMAT0000257 |
| ACAGCAGGCACAGACAGGCAGU | SEQ ID NO: 70 | hsa-mir-214 | MIMAT0000271 |
| AGGGGUGCUAUCUGUGAUUGA | SEQ ID NO: 71 | hsa-mir-342-5p | MIMAT0004694 |
| ACUGGACUUGGAGUCAGAAGG | SEQ ID NO: 72 | hsa-mir-378 | MIMAT0000732 |

The altered expression of these miRNAs was determined as shown in the Examples by performing a genome wide miRNA micro-array on tissue samples from end-stage human heart failure with dilated cardiomyopathy (DCM). Of the 288 human miRNA analyzed from 50 heart failure and 20 normal controls using methods of the present described below, the above noted nine miRNAs were substantially altered in heart failure and thus have substantial functional and diagnostic significance.

Of these miRNA, the expression level of hsa-mir-001, hsa-mir-007, hsa-mir-29b, and hsa-mir-378 were found to be down-regulated in a biological sample (e.g. myocardial tissue biopsy or PBMCs) from a subject with heart disease (e.g. heart failure) compared to a expression level of these miRNA in a healthy subject. Whereas, the expression level of has-mir-125b, hsa-mir-181b, hsa-mir-214, and hsa-mir-342 were found to be significantly up-regulated in biological samples (e.g. myocardial tissue biopsy or PBMCs) from subjects with heart disease (e.g. heart failure) compared to the expression levels of these miRNA in healthy subjects. Compared to the other miRNAs identified, the expressions levels of has-mir-007 and hsa-mir-378 were found to be substantially down-regulated in samples obtained from subjects with DCM. In one aspect of the method, the expression level of at least one of hsa-mir-007 and hsa-mir-378 is measured in the biological sample obtained from the subject and compared to a control expression level(s) to assess whether the subject has heart disease. In another aspect of the invention, the expression level of hsa-miRNA-1, has-miRNA-7, hsa-miRNA-29b, hsa-miRNA-125b, hsa-miRNA-145, hsa-miRNA-181b, has-miRNA-214, hsa-miRNA-342, and hsa-miRNA-378 are measured and compared to control expression levels to assess whether the subject has heart disease.

The measured expression levels can be used to generate a miRNA expression profile for a sample. It is specifically contemplated that miRNA expression profiles for subjects, particularly those suspected of having a particular form of heart disease, can be generated by evaluating any of or sets of the miRNAs discussed in this application. The miRNA profile that is generated from the patient will be one that provides information regarding the particular form of heart disease and/or the stage of disease progression.

In an aspect of the invention, a miRNA expression profile can be generated by steps that include: (a) labeling miRNA in the sample; (b) hybridizing miRNA to a number of probes, or amplifying a number of miRNA, and (c) determining miRNA hybridization to the probes or detecting miRNA amplification products, wherein a miRNA expression profile is generated. See U.S. Provisional Patent Application 60/575,743 and the U.S. Provisional Patent Application 60/649,584, and U.S. Patent Application Ser. No. 11/141,707, all of which are hereby incorporated by reference.

The methods can further comprise one or more of the steps including:

(a) obtaining a biological sample from the patient, (b) isolating nucleic acids from the sample, (c) labeling the nucleic acids isolated from the sample, and (d) hybridizing the labeled nucleic acids to one or more probes. Nucleic acids of the invention can include one or more nucleic acid comprising at least one segment having a sequence or complementary sequence of one or more of the miRNA sequences in Table 1. In certain aspects, the nucleic acids identify one or more miRNAs listed in Tables 1 and 2.

Certain embodiments of the invention include generating a miRNA expression profile of two or more miRNAs by using an amplification assay or a hybridization assay, a variety of which are well known to one of ordinary skill in the art. In certain aspects, an amplification assay can be a quantitative amplification assay, such as quantitative RT-PCR or the like. In still further aspects, a hybridization assay can include array hybridization assays or solution hybridization assays. The miRNA may be labeled from the sample and/or hybridizing the labeled miRNA to one or more miRNA probes (e.g. the probes included in Table 1 and Table 2.)

Nucleic acids, miRNA, and/or miRNA probes described herein, may be coupled to a support. Such supports are well known to those of ordinary skill in the art and include, but are not limited to glass, plastic, metal, or latex. Microarrays typically use coated glass as the solid support, in contrast to the nitrocellulose-based material of filter arrays. In particular aspects of the invention, the support can be planar or in the form of a bead or other geometric shapes or configurations known in the art.

In addition to the use of arrays and microarrays, it is contemplated that a number of different assays could be employed to generate miRNA expression profiles. Such assays include, but are not limited to, nucleic amplification, polymerase chain reaction, quantitative PCR, RT-PCR, in situ hybridization, Northern hybridization, hybridization protection assay (HPA)(GenProbe), branched DNA (bDNA) assay (Chiron), rolling Circle amplification (RCA), single molecule hybridization detection (US Genomics), Invader assay (ThirdWave Technologies), and/or Bridge Litigation Assay (Genaco).

In another aspect of the present invention, the expression profile can be measured using a miRNA microarray. The miRNA microarray can include two or more miRNA specific probes is provided. The probes have two or more miRNA coding sequences corresponding to hsa-miRNA-1, hsa-miRNA-7, hsa-miRNA-29b, hsa-miRNA-125b, hsa-miRNA145, hsa-miRNA-181b, hsa-miRNA-214, hsa-miRNA-342, hsa-miRNA-378 and combinations thereof. In some embodiments, the miRNA microarray can be immobilized on a support. The support can be comprised of any suitable material known to those having skill in the art.

Representative methods and apparatus for preparing a microarray have been described, for example, in U.S. Pat. Nos. 5,143,854; 5,202,231; 5,242,974; 5,288,644; 5,324,633; 5,384,261; 5,405,783; 5,412,087; 5,424,186; 5,429,807; 5,432,049; 5,436,327; 5,445,934; 5,468,613; 5,470,710; 5,472,672; 5,492,806; 5,525,464; 5,503,980; 5,510,270; 5,525,464; 5,527,681; 5,529,756; 5,532,128; 5,545,531; 5,547,839; 5,554,501; 5,556,752; 5,561,071; 5,571,639; 5,580,726; 5,580,732; 5,593,839; 5,599,695; 5,599,672; 5,610;287; 5,624,711; 5,631,134; 5,639,603; 5,654,413; 5,658,734; 5,661,028; 5,665,547; 5,667,972; 5,695,940; 5,700,637; 5,744,305; 5,800,992; 5,807,522; 5,830,645; 5,837,196; 5,871,928; 5,847,219; 5,876,932; 5,919,626; 6,004,755; 6,087,102; 6,368,799; 6,383,749; 6,617,112; 6,638,717; 6,720,138, as well as WO 93/17126; WO 95/11995; WO 95/21265; WO 95/21944; WO 95/35505; WO 96/31622; WO 97/10365; WO 97/27317; WO 99/35505; WO 09923256; WO 09936760; W00138580; WO 0168255; W003020898; WO 03040410; WO 03053586; WO 03087297; WO 03091426; W003100012; WO 04020085; WO 04027093; EP 373 203; EP 785 280; EP 799 897 and UK 8 803 000; the disclosures of which are all herein incorporated by reference.

It is contemplated that the arrays can be high density arrays, such that they contain 2, 20, 25, 50, 80, 100 or more different probes. It is contemplated that they may contain 1000, 16,000, 65,000, 250,000 or 1,000,000 or more different probes. The probes can be directed to targets in one or more different organisms or cell types. The oligonucleotide probes range from 5 to 50, 5 to 45, 10to 40, 9 to 34, or 15 to 40 nucleotides in length in some embodiments. In certain embodiments, the oligonucleotide probes are 5, 10, 15, 20 to 20, 25, 30, 35, 40 nucleotides in length including all integers and ranges there between.

The arrays may vary in a number of different ways, including average probe length, sequence or types of probes, nature of bond between the probe and the array surface, e.g. covalent or non-covalent, and the like. The labeling and screening methods described herein and the arrays are not limited in its utility with respect to any parameter except that the probes detect miRNA; consequently, methods and compositions may be used with a variety of different types of miRNA arrays.

The location and sequence of each different probe sequence in the array are generally known. Moreover, the large number of different probes can occupy a relatively small area providing a high density array having a probe density of generally greater than about 60, 100, 600, 1000, 5,000, 10,000, 40,000, 100,000, or 400,000 different oligonucleotide probes per cm². The surface area of the array can be about or less than about 1, 1.6,2,3,4,5,6, 7,8,9, or 10 cm².

Moreover, a person of ordinary skill in the art could readily analyze data generated using an array. Such protocols are disclosed above, and include information found in WO 9743450; WO 03023058; WO 03022421; WO 03029485; WO 03067217; WO 03066906; WO 03076928; WO 03093810; WO 03100448A1, all of which are specifically incorporated by reference.

After an array or a set of miRNA probes is prepared, and/or the miRNA in the sample or miRNA probe is labeled, the population of target nucleic acids in a sample is contacted with the array or probes under hybridization conditions, where such conditions can be adjusted, as desired, to provide for an optimum level of specificity in view of the particular assay being performed. Suitable hybridization conditions are well known to those of skill in the art and reviewed in Sambrook et al. (2001) and WO 95/21944. Of particular interest in many embodiments is the use of stringent conditions during hybridization. Stringent conditions are known to those of skill in the art.

The small surface area of the array permits uniform hybridization conditions, such as temperature regulation and salt content. Moreover, because of the small area occupied by the high density arrays, hybridization may be carried out in extremely small fluid volumes (e.g. about 250 μl or less, including volumes of about or less than about 5, 10, 25, 50, 60, 70, 80, 90, 100 μl, or any range derivable therein). In small volumes, hybridization may proceed very rapidly.

Other examples of “microarrays” or colloquially “chips” that can be used for detecting miRNA in accordance with the present invention are generally described in the art, for example, U.S. Pat. Nos. 5,143,854, 5,445,934, 5,744,305, 5,677,195, 6,040,193, 5,424,186 and Fodor et al., (1991), each of which is incorporated by reference in its entirety for all purposes. Techniques for the synthesis of these arrays using mechanical synthesis methods are described in, e.g. U.S. Pat. No. 5,384,261, incorporated herein by reference in its entirety for all purposes. Although a planar array surface is used in certain aspects, the array may be fabricated on a surface of virtually any shape or even a multiplicity of surfaces. Arrays may be nucleic acids on beads, gels, polymeric surfaces, fibers such as fiber optics, glass or any other appropriate substrate, see U.S. Pat. Nos. 5,770,358, 5,789,162, 5,708,153,6,040,193 and 5,800,992, which are hereby incorporated in their entirety for all purposes. Arrays may be packaged in such a manner as to allow for diagnostics or other manipulation of an all inclusive device, see for example, U.S. Pat. Nos. 5,856,174 and 5,922,591 incorporated in their entirety by reference for all purposes. See also U.S. patent application Ser. No. 09/545,207, filed Apr. 7, 2000 for additional information concerning arrays, their manufacture, and their characteristics, which is incorporated by reference in its entirety for all purposes.

The miRNA microarrays of the present invention can be used to evaluate samples with respect to heart disease. It is specifically contemplated that the invention can be used to evaluate differences between stages or sub-classifications of heart disease, such as between early or late stage heart failure.

In accordance with yet another aspect of the present invention, a kit for generating a miRNA profile for a sample is provided. The kit includes two of more miRNA probes having two or more miRNA coding sequences corresponding to sequences identified in TABLE 1 and TABLE 2, and reagents for labeling miRNA in the sample in a suitable container means.

In some embodiments, kits can be used to measure two or more miRNA molecules. Kits may comprise components, which may be individually packaged or placed in a container, such as a tube, bottle, vial, syringe, or other suitable container means. Individual components may also be provided in a kit in concentrated amounts; in some embodiments, a component is provided individually in the same concentration as it would be in a solution with other components. Concentrations of components may be provided as 1×, 2×, 5×, 10×, or 20× or more.

Kits for using miRNA probes, synthetic miRNAs, and/or non-synthetic miRNAs of the invention described herein for prognostic, or diagnostic applications are included as part of the invention. Specifically contemplated are any such molecules corresponding to any miRNA whose expression levels are reported to be significantly altered in heart failure compared to non-failing controls, such as those discussed herein. In certain aspects, negative and/or positive control synthetic miRNAs are included in some kit embodiments.

Certain embodiments are directed to a kit for assessment of heart disease in a subject by generating the miRNA expression profile of a sample comprising, in suitable container means, two or more miRNA hybridization or amplification reagents comprising two or more of hsa-miRNA-1, hsa-miRNA-7, hsa-miRNA-29b, hsa-miRNA-125b, has-miRNA145, hsa-miRNA-181b, hsa-miRNA-214, hsa-miRNA-342, or hsa-miRNA-378. The kit can comprise reagents for labeling miRNA in a sample and/or miRNA hybridization reagents. The miRNA hybridization reagents typically comprise hybridization probes. miRNA amplification reagents include, but are not limited to amplification primers.

In accordance with the provisions of the patent statutes, the principle and mode of operation of this invention have been explained and illustrated in its preferred embodiment. However, it must be understood that this invention may be practiced otherwise than as specifically explained and illustrated without departing from its spirit or scope.

The following examples are included to demonstrate embodiments of the invention. It should be appreciated by those of skill in the art that the techniques disclosed in the examples which follow represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### EXAMPLES

### Example 1

### End-Stage Heart Failure MicroRNA Fingerprint Indicates Regulation of Specific Cardiovascular Signaling Networks

In this example we a) measured the changes in expression of 288 human miRNAs using a micro-array hybridization platform; b) used a independent set of human heart failure samples to validate the data set; c) through in silico strategy of bioinformatics delineated canonical/functional pathways regulated by genes that are potential targets of miRNAs in heart failure to accomplish global molecular network analysis; and d) finally, to validate the target genes used the available gene expression databases to map the significantly changed miRNAs onto the global molecular networks. Using this strategy, multiple genes and pathways involved in this complex pathology can be visualized simultaneously allowing for a systems biology approach to human heart failure and potential novel therapeutics.

### Materials and Methods

### Patient Samples

Tissue from the left ventricular free wall was obtained from explanted hearts of transplant recipients at the Cleveland Clinic with a diagnosis of DCM. The non-failing control hearts were obtained from unmatched donors whose hearts were not suitable to transplantation despite normal ventricular structure and function as measured by echocardiography. The hearts were arrested and transported in ice-cold, oxygenated cardioplegic solution. Once in the lab the tissue was flash frozen in liquid N2 and stored at −80° C.

### RNA Isolation

100 mg of left ventricular tissue was homogenized using TRIZOL (INVITROGEN) reagent and the homogenized samples was incubated in room temperature for 5 min. Chloroform was added to the samples, vigorously mixed and incubated at room temperature for 5 min. Following incubation, the samples were centrifuged at 12,000×g for 15 min at 4° C. RNA was precipitated from the aqueous phase by addition of isopropranol to the in a fresh tube containing the supernatant aqueous phase. The integrity of the RNA was tested by spectroscopic analysis and by running all the isolated samples on a denaturing formaldehyde gel.

### Target Preparation and Array Hybridization

Five mg of total RNA was added to biotinylated oligonucleotide primer. Following incubation first strand was synthesized using Superscript II RNAseH reverse transcriptase. After t synthesis of the first strand, the reaction was incubated at 65° C. to denature the RNA/DNA hybrids and degrade RNA templates. The labeled targets were then used for chip hybridization. Hybridization was carried out on the miRNA microarray (Ohio State Comprehensive Cancer Centre, version 3.0) containing 627 probes for mature miRNA corresponding to 288 different human miRNAs spotted in quadruplicates. Often, more than one probe set is present for a given mature miRNA and there are quadruplicate probes corresponding to most precursor miRNAs. The detection of biotin-containing transcripts were carried out by streptavidin-Alexa Fluor 647 conjugate and scanned images (Axon 4000B) were quantified using the GenePix 6.0 software (Axon Instruments).

Computational Analysis of miRNA Microarray Data and miRNA Target Prediction:

Average values of tile replicate spots for each miRNA were background subtracted, normalized and subjected to further analysis. Global median normalization and Lowess normalization of the heart microarray data was carried out using the BRB ArrayTools. The probes with over 70% missing data were excluded from further analysis. Differentially expressed miRNAs between control and dilated cardiomyopathic samples were identified by using t test procedure within significance analysis of microarrays (SAM). Furthermore, Lowess normalization was carried out to analyze differentially expressed miRNAs in control versus diseased state. Following the identification of differentially expressing miRNAs, the predicted targets for these differentially expressed miRNAs were identified using TargetScan and PictTar databases. The need for analysis using the different databases was based on the need to encompass all the potential targets as they are built slightly different algorithms. We have used results of predicated targets from TargetScan database to carry out pathways and network analysis.

### Network Analysis

A data set containing genes and the corresponding expression values was uploaded into the INGENUITY PATHWAYS ANALYSIS NETWORK application. The dataset molecules of our interest (predicted targets of altered miRNA) which interact with other molecules in the Ingenuity's knowledge base are identified as network eligible molecules. Network eligible molecules serve as “seeds” for generating networks. Network eligible molecules arc combined into networks that maximize their connectivity in the Ingenuity's knowledge base. A defined network is limited to a maximum of 35 molecules and the additional molecules from the Ingenuity's knowledge base are used to connect networks resulting in large merged networks.

Predicated targets for the altered miRNA gene was mapped to the corresponding gene object in the Ingenuity knowledge base. A specific value for cut-off of 1.5 was set to identify genes whose expression was significantly differentially regulated. These genes, called focus genes, were overlaid into a global molecular network developed from the information contained in the Ingenuity Pathways Knowledge Base. Networks of these focus genes were then algorithmically generated based on their connectivity. A network pathway is a graphical representation of the molecular relationships between genes/gene products. Genes or gene products are represented as nodes, and the biological relationship between two nodes is represented as an edge (line). All edges are supported by at least one reference from literature, textbook or from canonical information stored in the Ingenuity Pathways Knowledge base. Nodes are displayed using various shapes that represent the functional class or the gene product. Edges are displayed with various labels that describe the nature of the relationship between the nodes (i.e., P for phosphorylation, T for transcription etc.).

### Canonical Pathway and Functional Analysis

Canonical pathway analysis was carried out using the Ingenuity Pathways Analysis (IPA) library of canonical pathways by uploading the data set of predicted targets for the significantly altered miRNAs in DCM to the IPA server. These target genes (focus genes) were analyzed for over-representative canonical pathways in control and diseased human samples. Significance of association between these genes and the canonical pathway was measured in two ways: a) A ratio of the number of genes from the data set that map to the pathway divided by total number of genes that map to the canonical pathway is displayed and b) Fischer's exact test was used to calculate a p value determining the probability that the association between the genes and the canonical pathway is explained by chance alone. Functional analysis of a network identified the biological functions that were most significant to the genes in the network. The network genes associated biological function/disease state in the Ingenuity knowledge base was considered for analysis. The network score is based on the hyper-geometric distribution and is calculated with the right-tailed Fisher's Exact Test and is represented as a negative log of this p-value. For example, a network of 35 molecules has a Fisher Exact Test p-value of 1×10−6, the network's score=−log (p-value)=6.18.

### Cardiac Microarray Expression Database

To test for changes in expression in the target gene sets, microarray data from cardio-genomics database for normal and idiopathic heart failure were used. The raw data were reprocessed using the GCRMA algorithm which is a three step function implemented in the GCRMA package (version 2.8.1) of the Bio-conductor open source library (version 2.5.0). Steps include correction of perfect match probe set expression signals for optical noise and non-specific binding using probe sequence information, followed by quantile normalization to smooth individual probes intensities. Finally, expression values were summarized by the robust multi-chip model fit using median polish. The summarized probe set expression values were subsequently fit to a linear correlation analysis model.

### Results

To get a global expression pattern for the miRNAs in the end-stage heart failure, mRNA was isolated from 70 patient samples (non-failing (20) and end-stage heart failure (50) with the diagnosis of DCM). The patient characteristics are summarized in Table 3. All failing hearts had left ventricular ejection fractions<15% and all the non-failing had ejection fraction>61% (Table 3). Mean age for the patients with DCM was 51±2 yrs and for non-failing was 54±1 yrs. The average age of the patients was 52.5±3 yrs and was gender and race independent. They all had relatively normal ventricular function as measured by echocardiography with no associated visible characteristic dysfunction as measured by echocardiography. Furthermore, a majority of the patients were on some form of inotropic and/or vassopressor support (Table 3).

TABLE 3
Patient Demographics
A. Non-Failing Hearts (n = 20)

| Age | Sex | Race | EF | Cause of Death | Drug Therapy |
|---|---|---|---|---|---|
| 51 ± 2 | 11F, | 19W, | 61 ± 2 | 14CVA, | Acute-NE, DA, |
| | 9M | 1B | | 2MVA, | Other |
| | | | | 1 GSW, | Chronic-“HTM |
| | | | | 1Trauma, | meds” |
| | | | | 1 Anoxia | |

B. Failing Hearts (n = 50)

| Age | Sex | Race | EF | Diagnosis | Drug Therapy |
|---|---|---|---|---|---|
| 54 ± 1 | 17F, | 42W, | 15 ± 1 | 50 DCM | DIG, DOB, AMIO, |
| | 33M | 8B | | | ACEI, BB |

Abbreviations:
Non-Failing Hearts: F = female, M = Male, W = White, B = Black, EF = left ventricular ejection fraction measured prior to explant; CVA = cerebrovascular accident, MVA = motor vehicle accident, GSW = gunshot wound;
Drug Therapy Acute = treatment in the emergency room or intensive care unit prior to brain death: NE = norepinephrine (n = 9), DA = dopamine (n = 12), OTHER = epinephrine, pitressin, phenylephrine, labetolol, lisinopril (n = 1 or 2);
Drug Therapy Chronic = drugs taken by patients prior to admission, as reported by family members (n = 6)
Failing Hearts:-F = female, M = Male, W = White, B = Black, EF = left ventricular ejection fraction measured prior to explants; DCM = dilated cardiomyopathy (pre-transplant diagnosis);
Drug Therapy lists those drugs taken by over 25% of patients in the group, DIG = digoxin, DOB-dobutamino, AMIO = amiodarone, ACEI-antiotensin converting enzyme inhibitor (usually lisinopril), BB = beta adrenergic blocker (metoprolol or carvedilol).

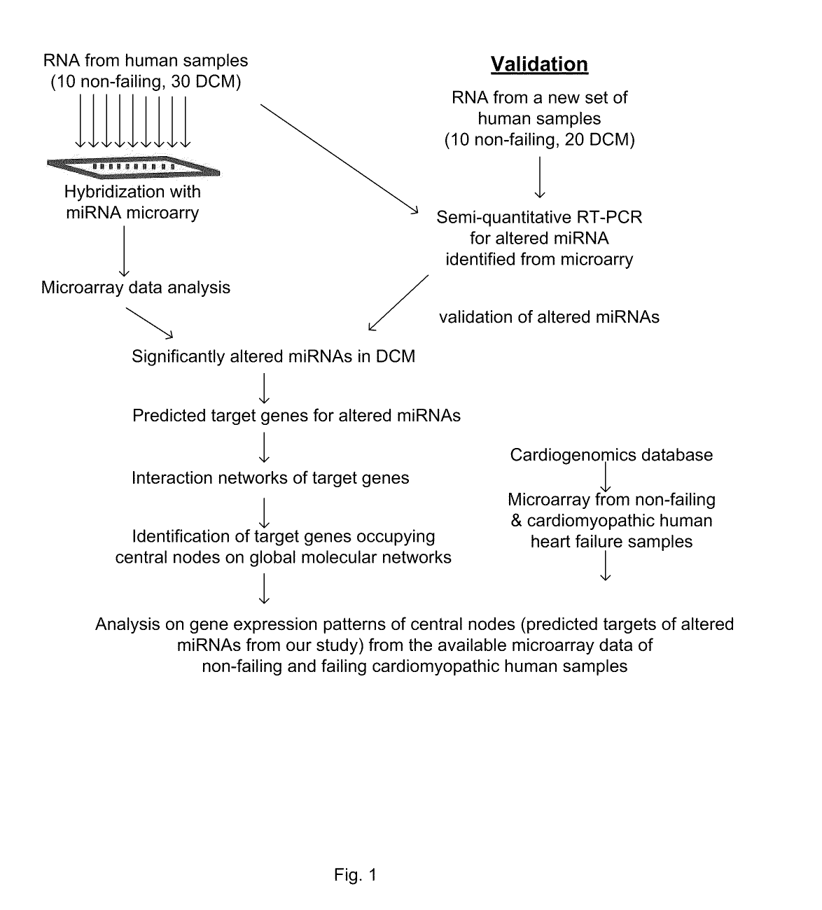

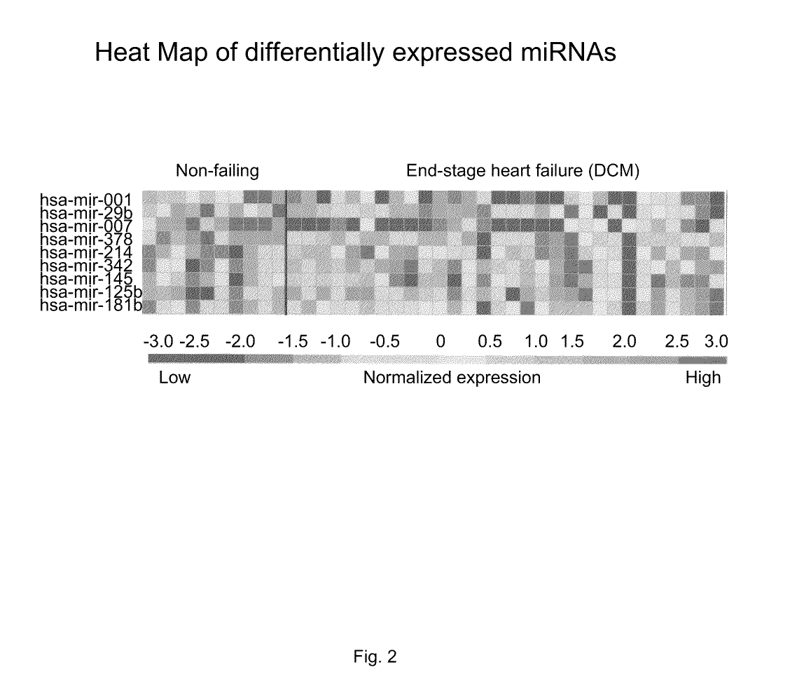

To test whether end-stage DCM is associated with a distinctive miRNA signature, we followed experimental plan described in FIG. 1. RNA from 10 non-failing and 30 end-stage dilated cardiomyopathic (DCM) patient samples were used to hybridize with the miRNA microarray. RNA from each patient was used an independent sample and therefore the miRNA microarray data set obtained is a unique expression profile for each individual patient sample. We have used a custom microarray platform containing 5760 miRNA probes (including 627 probes for 288 human miRNAs) that is well established and validated by previous studies 14 to evaluate miRNA expression profiles in end-stage dilated cardiomyopathy. To identify miRNAs differentially expressed in the failing versus the non-failing, the data from quantitative expression was normalized using global median and lowess normalization methods (see methods). Following these iterative processes of normalization, expression of nine miRNAs out of 288 different miRNAs were found to be significantly different between non-failing and DCM samples. The miRNAs hsa-mir-001 (p<0.00005), hsa-mir-29b (p<0.0087), hsa-mir-007 (p<0.0086) and hsa-mir-378 (p<0.0055) were significantly down-regulated in the DCM samples compared to non-failing controls. In contrast, miRNAs hsa-mir-214 (p<0.0001), hsa-mir-342 (p<0.0004), hsa-mir-145 (p<0.009), hsa-mir-125b (p<0.078) and hsa-mir-181b (p<0.0047) were significantly upregulated in DCM compared to non-failing controls. The expression values of these nine miRNA probes for all the individual patient samples are shown in the heat map (FIG. 2). Despite the individual patient variability to each of the miRNA probes, the heat map clearly demonstrates a distinctive pattern for specific miRNA expression associated with DCM.

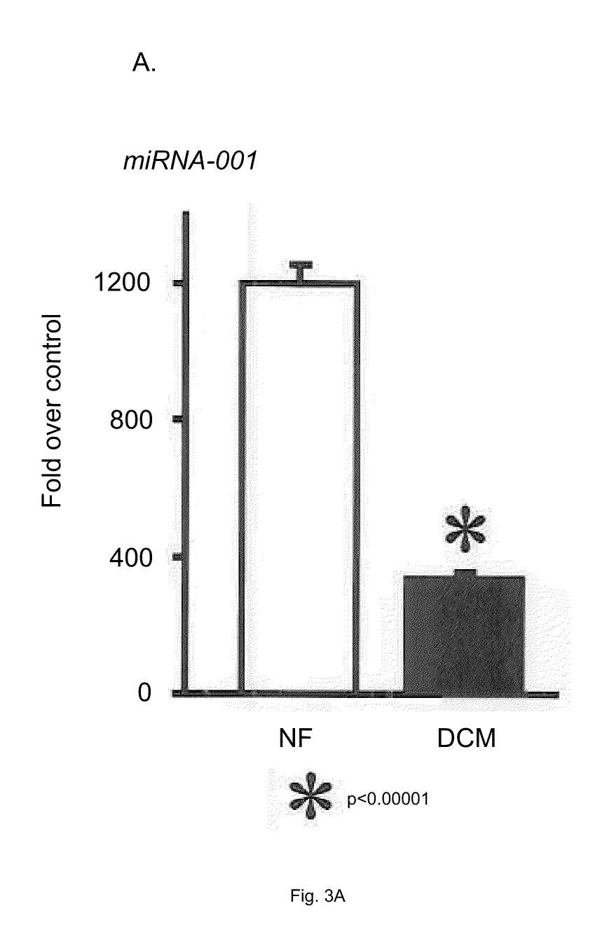

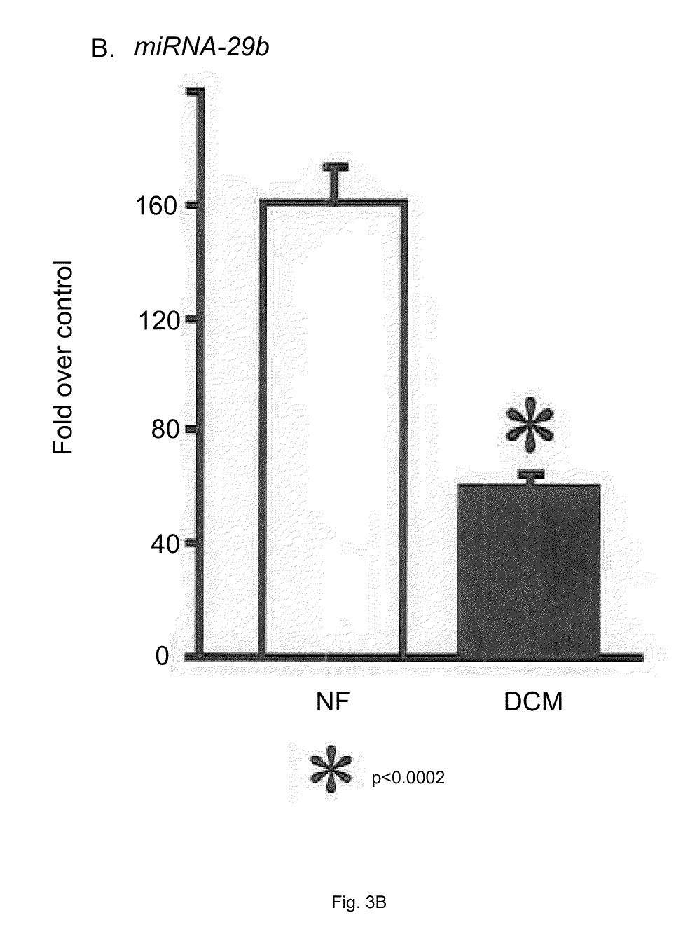

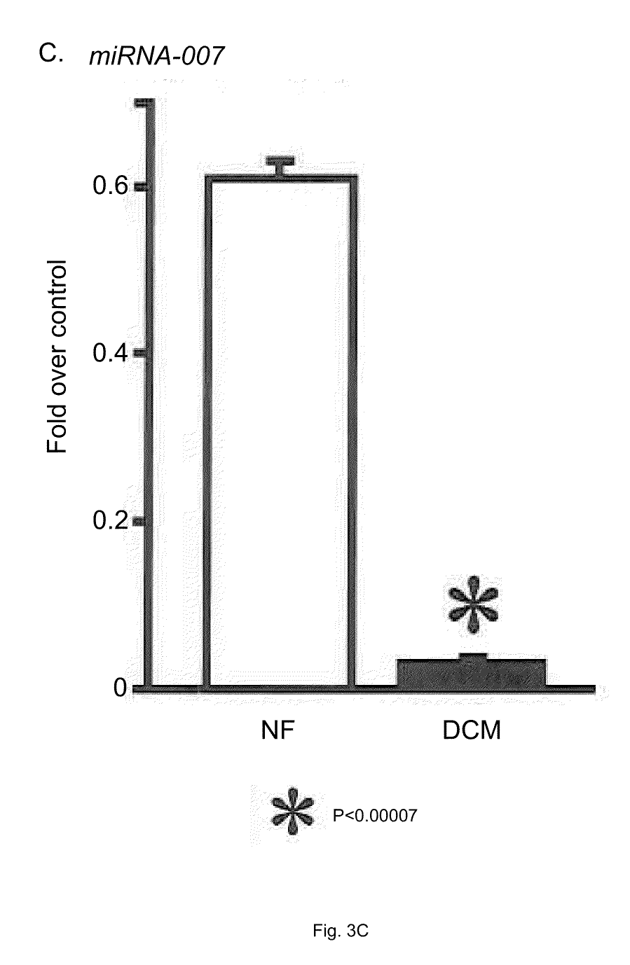

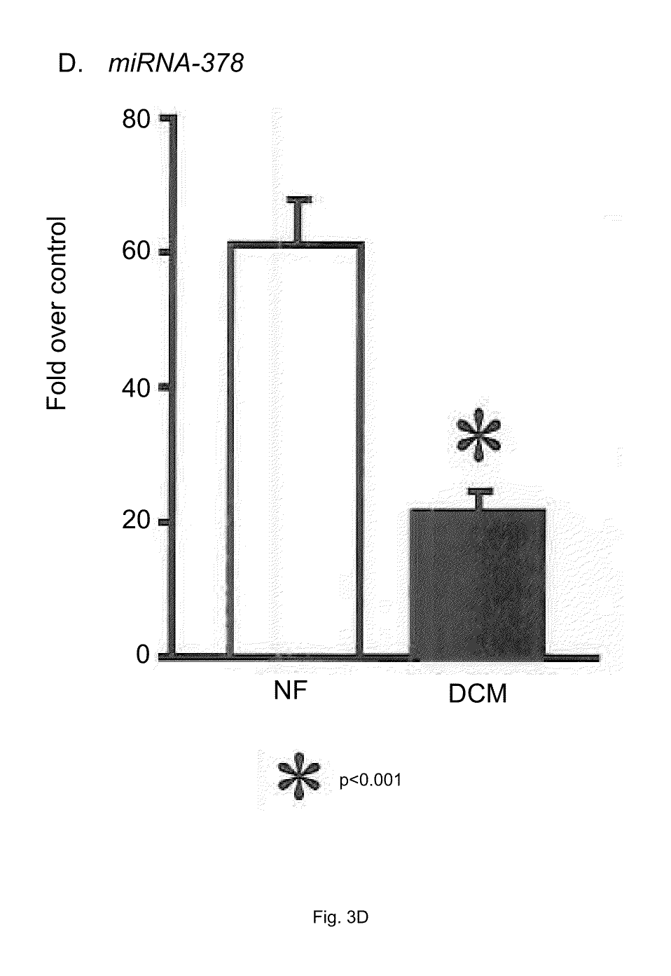

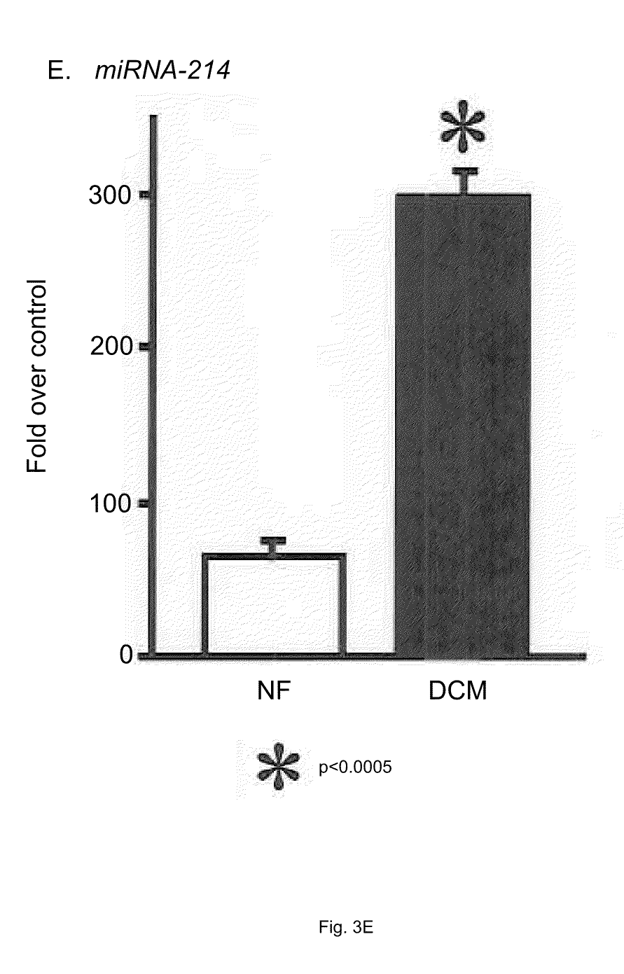

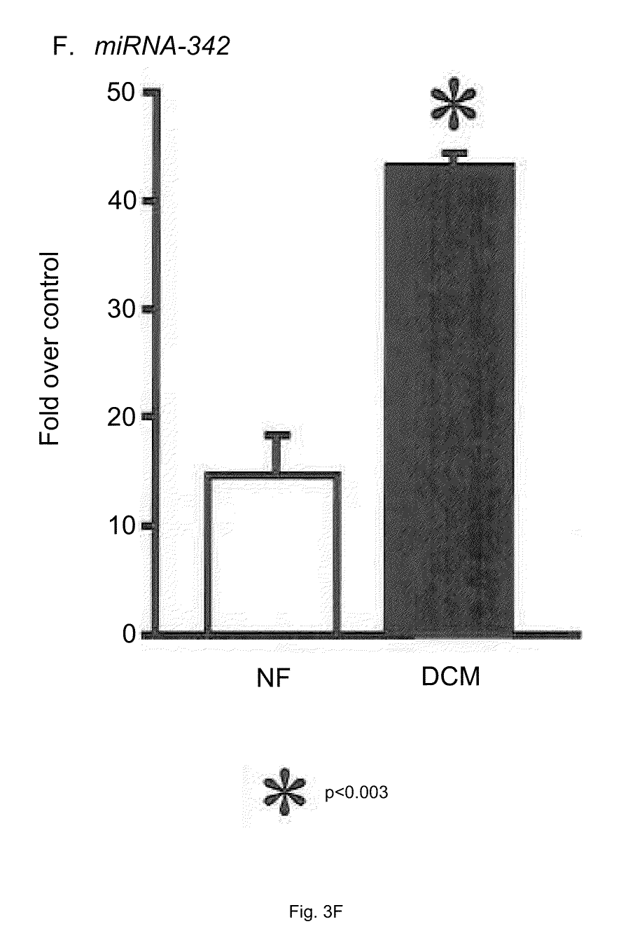

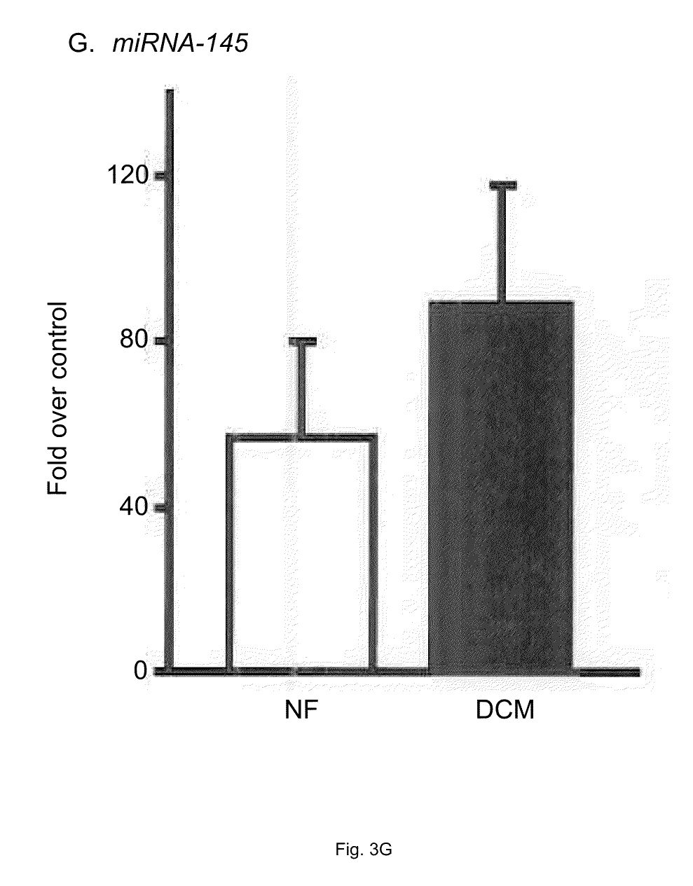

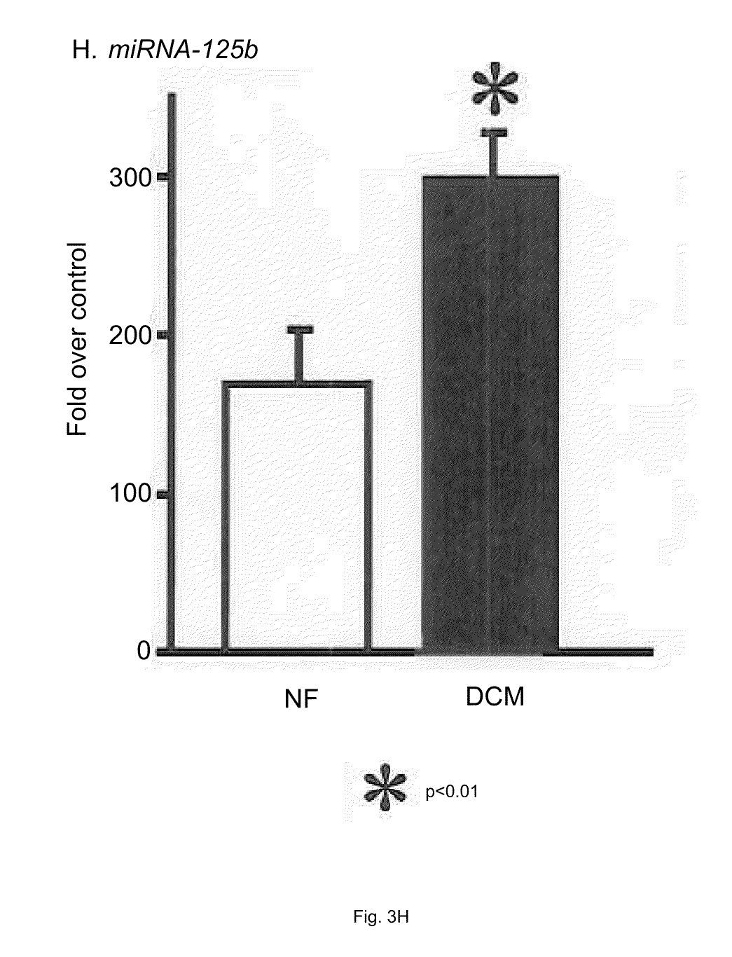

To test whether the same set of miRNAs can be validated, we used a new independent set of patient samples (10 non failing and 20 DCM). To validate these miRNAs, RNA from a new set of samples were isolated and subjected to RT-PCR using specific primers for the set of miRNAs. As internal control we used both 18S and U6. Our studies found that U6 gave more consistent results compared to 18S and therefore the miRNA data has been normalized to U6 values. RT-PCR from the new set of human patient samples showed that the differential expression of all the identified miRNAs could be validated except hsa-mir-145. To further confirm these results, we also carried out RT-PCR on the RNA from the samples that were first used for the miRNA microarray. Consistently, these studies also showed that differential expression of miRNAs could be validated except for hsa-mir-145. The RT-PCR data from the two independent sets of human samples consistently showed the same results and therefore it was appropriate to pool the data (20 non-failing and 50 dilated cardiomyopathy human patient samples) from the two analyses (FIG. 3). The results from the pooled data shows significant downregulation of hsa-miRNAs 1 (p<0.00001), 29b (p<0.0002), 7 (p<0.00007), 378 (p<0.001) and upregulation of has-miRNAs 214 (p<0.0005), 342 (p<0.003), 125b (p<0.01), 181b (p<0.005) validating the microarray studies (FIG. 3). Critically, hsa-miRNA-145 only showed a trend towards higher expression in DCM but did reach a level of significance compared to non-failing samples (FIG. 3g). Taken together, our data demonstrate that end-stage DCM has a specific miRNA signature that can be consistently revalidated in a large number of samples Importantly, our studies have identified novel miRNAs, hsa-miRNA-7 and hsa-miRNA-378, are significantly downregulated in DCM that may have critical roles in the pathophysiology of heart failure.

Altered miRNAs are Significantly Associated with Specific Canonical and Functional Pathways:

Since alteration in these miRNAs occur simultaneously modulating their respective targets, the global effect would be a sum total of effects coordinated by individual miRNAs. Analysis shows that a total of 1785 genes are predicted targets for the eight differentially expressed miRNAs in DCM.

To evaluate functional consequences based on combinatorial effect of the predicted targets of miRNAs an unbiased computational approach was taken using Ingenuity Pathway Knowledge base. The overrepresented canonical functional network with highest level of significance was cardiovascular system development and function (Table 4a). Identification of this network is entirely consistent with a role of the predicated targets of the altered miRNAs cardiovascular disease. Representation of these network eligible molecules in cellular and molecular functions such as cell signaling (p<5.91e−146) (Table 4b), gene expression (p<1.67e−126) (Table 4b), cell death (p<4.55e−93) (Table 2b) is highly significant and is further consistent with the documented dysregulation of these events and pathways in DCM.

TABLE 4A

| Top Associated Network Functions | Score |
|---|---|
| Cardiovascular System Development & Function, | 39 |
| Cell Signaling, Protein Degradation | |
| Gene Expression, Cell Signaling, Cellular Development | 35 |
| Gene Expression, Cellular Assembly & Organization, | 35 |
| Cellular Compromise | |
| Cell Signaling, Molecular Transport, Cell Death | 33 |
| Cell Signaling, Nervous System Development and Function, | 33 |
| Cell Morphology | |

TABLE 4B

| Molecular and Cellular Functions | p-value | # Molecules |
|---|---|---|
| Cell Signaling | 5.91E−146 | 368 |
| Gene Expression | 1.67E−126 | 254 |
| Cell Death | 4.55E−93 | 245 |
| Cellular Growth & Proliferation | 8.94E−84 | 273 |
| Cellular Development | 6.71E−77 | 229 |

Predicted Targets of Altered miRNAs Associate with Diverse Signaling Networks:

Of a total of 1785 predicated targets, 1716 could be mapped to signaling networks (see methods, definition of network) in the Ingenuity Pathways Knowledge Base network algorithm (IPATM) and 995 predicated targets were found to be network eligible (see methods for eligibility). The 995 network eligible candidates mapped to 43 networks, which are predicted to be involved in the cross-talk with the peripheral molecules bridging different networks resulting in the phenotype.

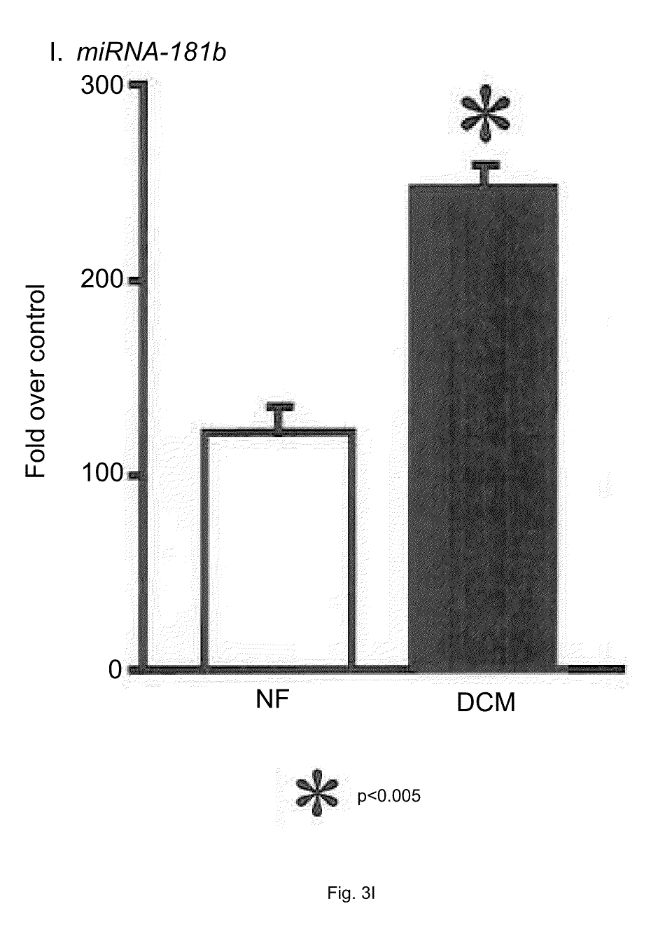

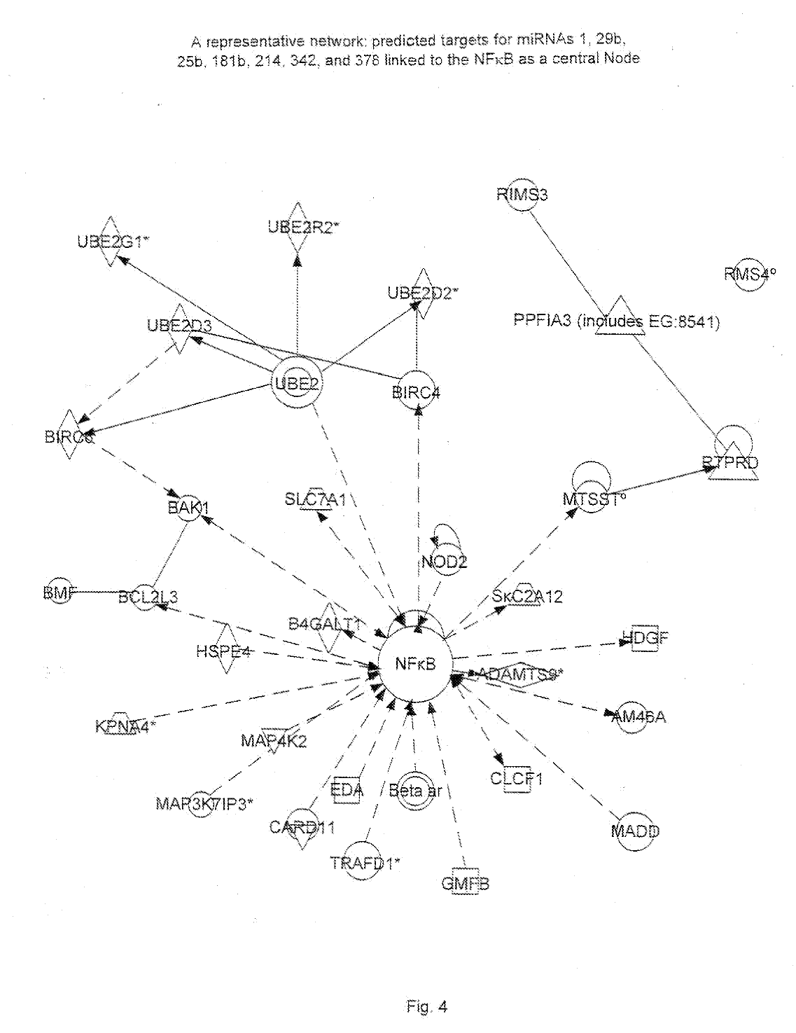

A representative network with NFkB, a known mediator in cardiac dysfunctionl9 as a central node, is shown in FIG. 4 wherein many of the network members that feed into NFkB are targets for the miRNAs 1, 29b, 125b, 181b, 214, 342 and 378. As individual miRNA acts on each target, the net effect on the node would be the collective effect of all the members that feed into the central node NFkB (FIG. 4). Based on these targets we predict that the complete NFkB regulatory signaling network (FIG. 4) would be significantly downregulated in DCM since a number of molecules in this network arc predicted targets for upregulated miRNAs 125b, 181b, 214 and 243. Although FIG. 4 predicts one specific network with NFkB as a central node for various miRNA, each network does not affect a physiological process in isolation and global regulation would involve integrative cross-talk among the networks to mediate the disease phenotype. In order to test for such a cross-talk between networks, we have used Ingenuity Pathways Analysis algorithm for overlaying and merging networking. The analysis of networks involved in top molecular and cellular functions associated with DCM (Table 4b) showed that out of the 75 networks, only 43 are predicted to be involved in the cross-talk with the peripheral molecules bridging different networks. A representation of the merged networks predicts that the represented pathways are specific for DCM; 32 networks were not incorporated into the network merge, suggesting they are not involved in cross-talk. Taken together this iterative analysis indicates that a specific set of pathways are operational which are associated with integrative connecting networks resulting in the manifestation of DCM phenotype.

We next analyzed whether nodes (see methods) of the various networks which are interconnected and overrepresented in DCM are predicted targets for the differentially expressed miRNAs in end-stage heart failure. IPA predicted nodal molecules on the various merged networks were analyzed for miRNA targets using the target prediction algorithms. This analysis shows that a significant number of nodal molecules are predicated targets of miRNA that are altered in DCM (Table 5). Some of the predicted molecules are potential targets for two or three miRNAs while some are targets for none. Since we have identified these nodal molecules in signaling networks that are constituted by over represented pathways as predicted targets (Table 4b), we analyzed whether these nodal molecules are altered in end-stage human dilated cardiomyopathy using the cardio-genomics expression data base (see methods). Many of the nodal molecules are significantly altered in end-stage heart failure (Table 6). To further relate the regulation of predicted targets by altered miRNAs in dilated cardiomyopathy, we have mapped the changes in the expression pattern of the nodal molecules to changes in miRNAs (Table 4, underline and bold represent down- and up-regulation respectively). Indeed, the data show reverse complimentary alterations in the levels of many of the predicated targets compared to our validated miRNAs expression profile e.g. MLL, STATS (Table 6). Interestingly, we also see parallel alterations for some of the nodal molecules and miRNAs e.g. MMPs, TIMP2 (Table 6) and in some cases no effect on the nodal molecules e.g. RBI, E2F3 (Table 6) suggesting the miRNA is a component of the complex regulation of signaling during pathophysiology of heart failure.

TABLE 5
Potential targets of validated miRNA occupying central nodes on networks
Merged Networks

| | |
|---|---|
| 1, 2, 31, 32, 38, 9 | 11, 15, 2, 21, 32, 36 |

| Nodal | Targeting | Nodal | Targeting |
|---|---|---|---|
| Molecules | has-mir | Molecules | has-mir |
| MLL | 342 | PDGFRB | — |
| RB1 | 7 | SP1 | 7 & 378 |
| CTBP1&2 | — | PDGFRA | — |
| STAT3 | 125b | STAT3 | 125b |
| E2F3 | 342 & 378 | RB1 | 7 |
| HISTONE3 | 1 | BCL2 | 181b |
| MITF | 378 | ERBB2 | 7 |
| NKκB | — | ACVR2B | 214 & 18b |
| TERT | — | MMP 2&11 | 125b |
| HDAC 4&9 | 29b | TIMP2 | 214 |
| COL1A2 | 7 & 342 | CDK6 | 214 |
| | | PROTO-CADHER | |

| | |
|---|---|
| 17, 20, 22, 32, 7, 8 | 27, 28, 31, 33, 39, 42, 44 |

| Nodal | Targeting | Nodal | Targeting |
|---|---|---|---|
| Molecules | has-mir | Molecules | has-mir |
| MDB2 | 7 & 125b | YWHAG | — |
| RARB | 1 29b | DUB | — |
| EZH2 | 378 | SMARCA4 | 1 |
| E2F3 | 342 & 378 | | |
| PER1 | 214 | | |
| CSF1 | 214 | | |
| CAMK B&D | — | | |
| ZNF 217 | — | | |
| CCND1 | 1 | | |
| CREB5 | 1 & 214 & 181b | | |

TABLE 6
Merged Networks

| | |
|---|---|
| 1, 2, 31, 32, 38, 9 | 11, 15, 2, 21, 32, 36 |

| Nodal | Targeting | Nodal | Targeting |
|---|---|---|---|
| Molecules | hsa-mir | Molecules | hsa-mir |
| MLL | 342 | PDGFRB | — |
| RB1 | 7 | SP1 | 7 & 378 |
| CTBP1&2 | — | PDGFRA | — |
| STAT3 | 125b | STAT3 | 125b |
| E2F3 | 342 & 378 | RB1 | 7 |
| HISTONE3 | 1 | BCL2 | 181b |
| MITF | 378 | ERBB2 | 7 |
| NKκB | — | ACVR2B | 214 & 18b |
| TERT | — | MMP 2&11 | 125b |
| HDAC 4&9 | 29b | TIMP2 | 214 |
| COL1A2 | 7 & 342 | CDK6 | 214 |
| | | PROTO-CADHER | |

| | |
|---|---|
| 17, 20, 22, 32, 7, 8 | 27, 28, 31, 33, 39, 42, 44 |

| Nodal | Targeting | Nodal | Targeting |
|---|---|---|---|
| Molecules | hsa-mir | Molecules | hsa-mir |
| MDB2 | 7 & 125b | YWHAG | — |
| RARB | 1 & 29b | DUB | — |
| EZH2 | 378 | SMARCA4 | 1 |
| E2F3 | 342 & 378 | | |
| PER1 | 214 | | |
| CSF1 | 214 | | |
| CAMK B | — | | |
| ZNF 217 | — | | |
| CCND1 | 1 | | |
| CREB5 | 1 & 214 & 181b | | |

Bold = Down-regulation
Italicized = Up-Regulation

### Example 2

We have initiated an alternative, minimally invasive strategy, analysis of miRNAs in peripheral blood mononuclear cells (PBMCs) (containing lymphocytes and monocytes). miRNA expression patterns have been extensively studied in various human organs and tissues. However, there have been no investigations of miRNA expression in PBMCs. Hence, we propose to determine whether miRNAs expression patterns in PBMCs can provide a facile and precise approach to diagnose HF in its early stages and to monitor the response to therapies implemented to precent the progression of HF.

Numerous studies have attempted to use various blood components as predictors/indicators of cardiac function. Elevated white blood cell counts, enhanced phagocyte migration, abnormal levels of pro-inflammatory cytokines, soluble cytokine receptors, chemokines and peptide hormones have all been examined and have been shown to be changed in patients with chronic heart failure. Although these studies are interesting and valuable, none of the proposed biomarkers identify the early onset of cardiac dysfunction but are the manifestations of the changes that occur late in the process of maladaptive cardiac hypertrophy. The breadth and extent of changes observed in hypertrophic and failing hearts is entirely consistent with changes in miRNAs, which would be able to regulate multiple signaling molecules and pathways. Therefore, changes in miRNA profiles would be sentinels for initial pathological changes in the heart and could be monitored as a reliable diagnostic tool.

We have designed and begun a clinical study involving patients who are in the early stages of heart failure. Blood was drawn from patients at their first visit to the Cleveland Clinic who present symptoms of cardiac dysfunction (Stage I) with ejection fractions of 40-50%. The patients were placed on treatment to prevent progression of the disease and blood samples drawn after a six months on the medication. 30% of the patients did respond to beta-blockers, but have progressive dysfunction and are administered alternative treatments. This cohort of patients was an important group to analyze as it provided insights into alterations in miRNA in PBMCs with deterioration of cardiac function. All the patients were monitored for a total of 18 to 24 months with periodic blood draws every six months.

Blood sample were measured for the expression of the nine specific miRNAs in the heart tissue of HF patients (hsa-miRNA -1, -7, -29b, -125b, -145, -181b, -214, -342 and -378). Changes in miRNA expression was correlated with the ejection fractions of each patient at the time of the blood drawing.

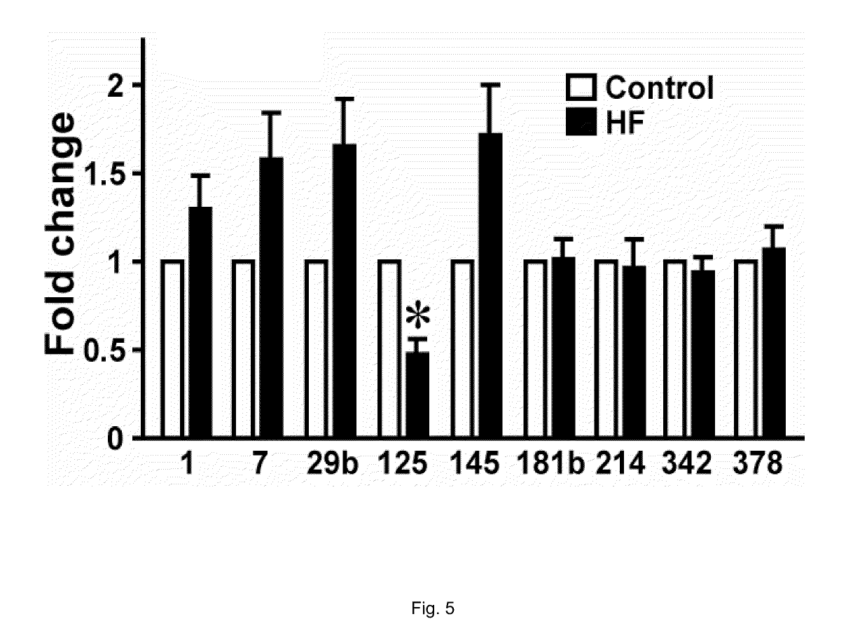

miRNA expression levels in PBMCs of 10 patients with symptoms of Stage I heart failure were analyzed and compared to ten healthy controls. RNA was isolated from PBMCs and real-time PCR reactions were carried for the specific set of miRNAs. The Litvak method of calculation was used to determine the fold changes of miRNAs in the samples. Analysis reveals a significant change in the expression of hsa-miRNA-125b and substantial alterations in three other miRNAs (hsa-miRNA-7, -29b and -145) (FIG. 5). Both increases and decreases in miRNA levels were expected and observed.

Despite the small sample size, our preliminary data strongly supports our hypothesis that there is a dynamic cross-talk between the circulating cells and cardiac function. Importantly, these data show that miRNAs are the initial molecular sensors for cardiac dysfunction. Our studies lay the foundation for a powerful diagnostic tool that could accomplish identification of cardiac dysfunction early in onset.

From the above description of the invention, those skilled in the art will perceive improvements, changes and modifications Such improvements, changes and modifications are within the skill of the art and are intended to be covered by the appended claims. All publications, patents, and patent applications cited in the present application are herein incorporated by reference in their entirety.

Having described the invention, the following is claimed:

1. A method for assessing heart disease in a subject comprising:
obtaining a biological sample from the subject that includes miRNA;
measuring the expression level of at least two miRNAs in the sample, the miRNAs selected from the group consisting of hsa-miRNA-1, hsa-miRNA-7, has-miRNA-29b, hsa-miRNA-125b, hsa-miRNA-145, hsa-miRNA-181b, hsa-miRNA-214, hsa-miRNA-342, hsa-miRNA-378 and combinations thereof; and
comparing the expression level of the at least two miRNAs to a control expression level, wherein a difference in the expression level of the sample from the subject and the control expression level is indicative of heart disease.

2. The method of claim 1, the sample comprising a myocardial tissue.

3. The method of claim 1, the sample comprising a blood sample.

4. The method of claim 3, the sample comprising peripheral blood mononuclear cells.

5. The method of claim 1, wherein a down-regulated expression level of at least one miRNA comprising hsa-mir-001, hsa-mir-007, hsa-mir-29b, or hsa-mir-378 compared to the control expression profile is indicative of heart disease.

6. The method of claim 1, wherein an up-regulated expression level of at least one miRNA comprising hsa-mir-125b, hsa-mir-181b, hsa-mir-214, or hsa-mir-342 compared to the control expression profile is indicative of heart disease.

7. The method of claim 1, the expression level of at least one of hsa-mir-007 and hsa-mir-378 being measured.

8. The method of claim 7, the expression level of hsa-mir-007 and hsa-mir-378 being measured.

9. The method of claim 7, the expression level of hsa-miRNA-1, hsa-miRNA-7, hsa-miRNA-29b, hsa-miRNA-125b, hsa-miRNA-145, hsa-miRNA-181b, hsa-miRNA-214, hsa-miRNA-342, and hsa-miRNA-378 being measured.

10. The method of claim 1, the expression level of at least two miRNAs being determined by an amplification assay.

11. The method of claim 1, the expression profile of at least two miRNAs being determined by a hybridization assay.

12. A method for assessing heart disease in a subject comprising:
obtaining a sample of peripheral blood mononuclear cells from the subject;
measuring expression levels of at least one miRNA associated with heart disease from the peripheral blood mononuclear cell sample, wherein the expression of the at least one miRNA from the peripheral blood mononuclear cell sample corresponds to miRNA expression level in the subject's myocardial tissue; and
comparing the expression levels of the measured miRNA to a control expression level, wherein a difference in the measured expression level and the control expression level is indicative of heart disease.

13. The method of claim 12, the expression levels miRNAs measured being selected from the group consisting of hsa-miRNA-1, hsa-miRNA-7, hsa-miRNA-29b, has-miRNA-125b, hsa-miRNA-145, hsa-miRNA-181b, hsa-miRNA-214, hsa-miRNA-342, hsa-miRNA-378 and combinations thereof.

14. The method of claim 12, wherein a down-regulated expression level of at least one miRNA comprising hsa-mir-001, hsa-mir-007, hsa-mir-29b, or hsa-mir-378 compared to the control expression profile is indicative of heart disease.

15. The method of claim 12, wherein an up-regulated expression level of at least one miRNA comprising hsa-mir-125b, hsa-mir-181b, hsa-mir-214, or hsa-mir-342 compared to the control expression profile is indicative of heart disease.

16. The method of claim 12, the expression level of at least one of hsa-mir-007 and hsa-mir-378 being measured.

17. The method of claim 16, the expression level of hsa-mir-007 and hsa-mir-378 being measured.

18. The method of claim 12, the expression level of hsa-miRNA-1, hsa-miRNA-7, hsa-miRNA-29b, hsa-miRNA-125b, hsa-miRNA-145, hsa-miRNA-181b, hsa-miRNA-214, hsa-miRNA-342, and hsa-miRNA-378 being measured.

19. The method of claim 12, the expression level of miRNAs being determined by an amplification assay.

20. A method for assessing heart disease in a subject comprising:
obtaining a sample of peripheral blood mononuclear cells from the subject;
measuring the expression level of at least two miRNAs in the sample, the miRNAs selected from the group consisting of hsa-miRNA-1, hsa-miRNA-7, has-miRNA-29b, hsa-miRNA-125b, hsa-miRNA-145, hsa-miRNA-181b, hsa-miRNA-214, has-miRNA-342, hsa-miRNA-378 and combinations thereof; and
comparing the expression level of the at least two or more miRNAs to a control expression level, wherein a difference in the expression level of the sample from the subject and the control expression level is indicative of heart disease.
